(19) United States
(12) Patent Application Publication
(10) Pub. No.: US 2025/0049499 A1
(43) Pub. Date: Feb. 13, 2025

(54) CATHETER WITH HYPOTUBE HAVING EXCHANGE JOINT OPENING

(71) Applicant: Medtronic Ireland Manufacturing Unlimited Company, Dublin (IE)

(72) Inventors: Jaclyn N. Kawwas, Philadelphia, PA (US); Jonathan Ashley Cope, Santa Rosa, CA (US); Alexandra C. Dotti, Rohnert Park, CA (US)

(21) Appl. No.: 18/722,446

(22) PCT Filed: Dec. 20, 2022
(86) PCT No.: PCT/EP2022/087052

§ 371 Date: Jun. 20, 2024

Related U.S. Application Data
(60) Provisional application No. 63/265,863, filed on Dec. 22, 2021.

(51) Int. Cl. A61B 18/14; A61B 18/00; A61M 25/00; A61M 25/01

(52) U.S. Cl. CPC A61B 18/1492; A61M 25/0013; A61M 25/0015; A61M 25/0029; A61M 25/0045; A61M 25/0054; A61M 25/01; A61B 2018/00083; A61M 2025/0034; A61M 2025/0035; A61M 2025/006; A61M 2025/018; A61M 2025/0183; A61M 2205/0238; A61M 2207/10

(57) ABSTRACT

A catheter includes a metal hypotube (328) and a polymer outer jacket over the metal hypotube, wherein the metal hypotube is configured to bend with varying flexibility along a length of the metal hypotube, and wherein the metal hypotube defines a rapid exchange joint opening in an intermediate portion of the metal hypotube. A method for manufacturing a catheter includes cutting a rapid exchange joint opening in an intermediate portion of a metal hypotube and a polymer jacket over the metal hypotube, wherein the rapid exchange joint opening facilitates formation of a rapid exchange joint, and the metal hypotube is configured to bend with varying flexibility along a length of the metal hypotube.

### TECHNICAL FIELD

The present technology is related to catheters, such as, for example, neuromodulation catheters including neuromodulation elements configured to deliver energy to nerves at or near a treatment location within a body lumen.

### BACKGROUND

The sympathetic nervous system (SNS) is a primarily involuntary bodily control system typically associated with stress responses. Fibers of the SNS extend through tissue in almost every organ system of the human body and can affect characteristics such as pupil diameter, gut motility, and urinary output. Such regulation can have adaptive utility in maintaining homeostasis or in preparing the body for rapid response to environmental factors. Chronic activation of the SNS, however, is a common maladaptive response that can drive the progression of many disease states. Excessive activation of the renal SNS, in particular, has been identified experimentally and in humans as a likely contributor to the complex pathophysiologies of hypertension, states of volume overload (e.g., heart failure), and progressive renal disease.

Sympathetic nerves of the kidneys terminate in the renal blood vessels, the juxtaglomerular apparatus, and the renal tubules, among other structures. Stimulation of the renal sympathetic nerves can cause, for example, increased renin release, increased sodium reabsorption, and reduced renal blood flow. These and other neural-regulated components of renal function are considerably stimulated in disease states characterized by heightened sympathetic tone. For example, reduced renal blood flow and glomerular filtration rate as a result of renal sympathetic efferent stimulation is likely a cornerstone of the loss of renal function in cardio-renal syndrome (i.e., renal dysfunction as a progressive complication of chronic heart failure). Pharmacologic strategies to thwart the consequences of renal sympathetic stimulation include centrally-acting sympatholytic drugs, beta blockers (e.g., to reduce renin release), angiotensin-converting enzyme inhibitors and receptor blockers (e.g., to block the action of angiotensin II and aldosterone activation consequent to renin release), and diuretics (e.g., to counter renal sympathetic mediated sodium and water retention). These pharmacologic strategies, however, have significant limitations including limited efficacy, compliance issues, side effects, and others.

### SUMMARY

The present technology is directed to catheters. Some examples are directed to neuromodulation catheters and techniques for assembling of neuromodulation catheters. A catheter (e.g., an ablation catheter) may include a number of different elements that take time to assemble. To simplify assembly and/or reduce assembly time, the catheter may include a single hypotube segment, where the hypotube segment also defines a rapid exchange joint opening in an intermediate portion of the hypotube segment. The hypotube segment may include a metal hypotube, which is configured to bend with varying flexibility along its length. For example, the metal hypotube may include a laser cut shaft, and the cuts (e.g., holes, slots, or the like) may vary in size, shape, or areal density along a length of the metal hypotube, wherein the cuts allow the metal hypotube to have varying flexibility along its length. In this way, a single hypotube may be used in place of multiple different structures, such as a proximal hypotube and an intermediate shaft, which may simplify assembly and reduce assembly time of the catheter.

In some examples, a catheter includes a metal hypotube and a polymer outer jacket over the metal hypotube, wherein the metal hypotube is configured to bend with varying flexibility along a length of the metal hypotube, and wherein the metal hypotube defines a rapid exchange joint opening in an intermediate portion of the metal hypotube.

In some examples, a method for manufacturing a catheter includes cutting a rapid exchange joint opening in an intermediate portion of a metal hypotube and a polymer jacket over the metal hypotube, wherein the rapid exchange joint opening facilitates formation of a rapid exchange joint, and the metal hypotube is configured to bend with varying flexibility along a length of the metal hypotube.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

Reference is made to the attached drawings, wherein elements having the same reference numeral designations represent similar elements throughout.

FIG. 1 is a partially schematic perspective view illustrating a therapeutic system including a neuromodulation catheter configured in accordance with an example of the present technology.

FIG. 2 is an exploded profile view of the neuromodulation catheter shown in FIG. 1.

FIG. 3 is an enlarged exploded profile view of portions of the neuromodulation catheter shown in FIG. 1 taken at respective locations designated in FIG. 2.

FIG. 4 is side view of an example metal hypotube with a rapid exchange joint opening, in accordance with one or more examples of the present disclosure.

FIG. 5 is a side view of an example metal hypotube with a guidewire tube passing through the rapid exchange joint opening, in accordance with one or more examples of the present disclosure.

FIG. 6 is a side view of an example metal hypotube and guidewire tube connected by reflowed polymer, in accordance with one or more examples of the present disclosure.

FIG. 7 is a side view of an example skived section of a guidewire tube, in accordance with one or more examples of the present disclosure.

FIG. 8 is a side view of an example metal hypotube with varying flexibility along a length of the metal hypotube, in accordance with one or more examples of the present disclosure.

FIG. 9 is an enlarged view of an example rapid exchange joint opening, in accordance with one or more examples of the present disclosure.

FIG. 10A is a cross sectional view of an example metal hypotube segment distal to a rapid exchange joint opening, including electrical leads and a guidewire lumen, in accordance with one or more examples of the present disclosure.

FIG. 10B is a cross sectional view of a metal hypotube segment proximal to a rapid exchange joint opening, including electrical leads extending through a polyimide sleeve, in accordance with one or more examples of the present disclosure.

FIG. 11 is a flow chart illustrating a method for assembling a neuromodulation catheter, in accordance with one or more examples of the present disclosure.

### DETAILED DESCRIPTION

The present technology is directed to neuromodulation catheters and techniques for assembling a neuromodulation catheter. Although examples are described primarily with respect to renal neuromodulation, a person having ordinary skill in the art reading this description will understand that the devices, systems, and methods described herein may be used for neuromodulation at any suitable location within a body of a patient, including intravascular locations. Exemplifying vascular locations include, for example, a renal artery, external iliac artery, internal iliac artery, internal pudendal artery, celiac artery, mesenteric artery, superior mesenteric artery, inferior mesenteric artery, hepatic artery, splenic artery, gastric artery, left gastric artery, pancreatic artery, uterine artery, ovarian artery, testicular artery, and/or their associated arterial branches, accessories, veins, etc.

As used herein, the terms “distal” and “proximal” define a position or direction with respect to the treating clinician or clinician's control device (e.g., a handle assembly). “Distal” or “distally” can refer to a position distant from or in a direction away from the clinician or clinician's control device. “Proximal” and “proximally” can refer to a position near or in a direction toward the clinician or clinician's control device.

Neuromodulation, such as denervation, may be used to modulate activity of one or more nerves and may be used to affect activity of the sympathetic nervous system (SNS). Renal neuromodulation, for example, may be used to modulate activity of one or more renal nerves and may be used to affect activity of the SNS. In renal neuromodulation, one or more therapeutic elements may be introduced near renal nerves located between an aorta and a kidney of a patient. In some examples, the one or more therapeutic elements may be carried by or attached to a catheter, and the catheter may be introduced intravascularly, e.g., into a renal artery via a brachial artery, femoral artery, or radial artery approach. In other examples, the one or more therapeutic elements may be introduced extravascularly, e.g., using a laparoscopic technique.

Neuromodulation may be accomplished using one or more of a variety of treatment modalities, including electrical stimulation, radio frequency (RF) energy, microwave energy, ultrasound energy, a chemical agent, thermal energy (e.g., cryoablation or direct heating) or the like. Although examples are described primarily with respect to the RF modality, a person having ordinary skill in the art reading this description will understand that the devices, systems, and methods described herein also may be used for neuromodulation employing other modalities, including those referenced herein. In some examples, an RF ablation system includes an RF generator configured to generate RF energy and deliver RF energy to tissue via one or more electrodes carried by a catheter and positioned within a lumen of a body of a patient. For example, the lumen may be a vessel, such as a vein or artery. In some examples, the lumen may be a renal artery, such as a main renal artery, an accessory renal artery, a branch vessel, or the like. The RF energy may heat tissue to which the RF energy is directed (which tissue includes one or more renal nerves) and modulate the activity of the one or more renal nerves.

A neuromodulation catheter (e.g., for performing renal denervation) may include a number of components for assembly. The higher the number of components, generally the longer it may take to assemble the neuromodulation catheter. Accordingly, it may be desirable to reduce the number of components for assembly of the neuromodulation catheter. Some neuromodulation catheters may include: a neuromodulation assembly including one or more electrodes, wherein each electrode is connected to one or more electrical leads (e.g., wires) that travel the length of the catheter back to a handle assembly; an intermediate shaft attached to a proximal end of the neuromodulation element; and a proximal hypotube attached to a proximal end of the intermediate shaft at or near a rapid exchange joint; a polyimide sleeve within a lumen defined by the proximal hypotube; and a guidewire tube positioned within the catheter distal to the rapid exchange joint, wherein a proximal portion of the guidewire tube exits the catheter at the rapid exchange joint.

In examples of the current disclosure, a neuromodulation catheter may include fewer components for assembly, and thus take less time to assemble. For example, a neuromodulation catheter (e.g., an RF ablation catheter) may include a single metal hypotube configured to bend with varying flexibility along a length of the metal hypotube that extends from a proximal handle assembly to a distal neuromodulation element. The metal hypotube also defines a rapid exchange joint opening in an intermediate portion of the hypotube. In examples of the current disclosure, instead of including an intermediate shaft and proximal hypotube as separate components, the catheter includes one metal hypotube with varying flexibility along a length of the metal hypotube. In this way, the metal hypotube may allow a neuromodulation catheter to navigate through varying tortious anatomy of the body, and may allow omission of a separate intermediate shaft. In addition, the rapid exchange joint that was defined at the attachment point between the intermediate shaft and the proximal hypotube may be replaced with a rapid exchange joint opening in an intermediate portion of the metal hypotube. In this, the assembly of neuromodulation catheters may be simpler and less time-intensive.

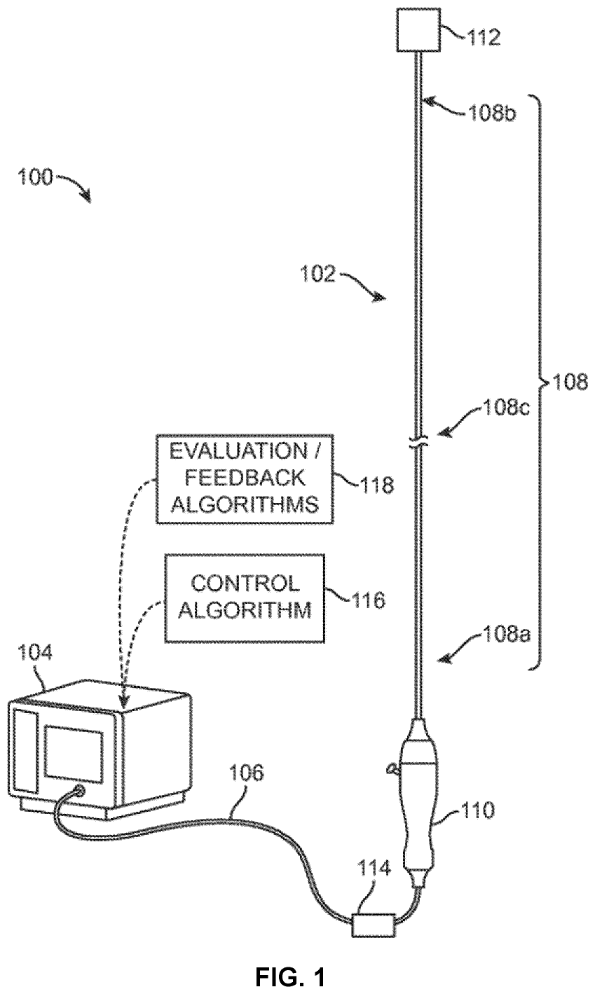

FIG. 1 is a partially schematic perspective view illustrating a therapeutic system 100 including a neuromodulation catheter configured in accordance with some examples of the present disclosure. Therapeutic system 100 includes a neuromodulation catheter 102, an RF generator 104, and a cable 106 extending between catheter 102 and RF generator 104. Neuromodulation catheter 102 includes an elongate shaft 108 (also referred to as an elongate body 108) having a proximal portion 108a, a distal portion 108b, and an intermediate portion 108c between proximal portion 108a and distal portion 108b. Neuromodulation catheter 102 may further include a handle 110 operably connected to elongate shaft 108 via proximal portion 108a and a neuromodulation element 112 (shown schematically in FIG. 1) that is part of or attached to distal portion 108b. Elongate shaft 108 is configured to locate the neuromodulation element 112 at a treatment location within or otherwise proximate to a body lumen (e.g., a blood vessel, a duct, an airway, or another naturally occurring lumen within the human body). In some examples, elongate shaft 108 is configured to locate neuromodulation element 112 at an intraluminal (e.g., intravascular) location. Neuromodulation element 112 may be configured to provide or support a neuromodulation treatment at the treatment location. Elongate shaft 108 and neuromodulation element 112 may measure 2, 3, 4, 5, 6, or 7 French or other suitable sizes.

Intraluminal delivery of neuromodulation catheter 102 may include percutaneously inserting a guidewire (not shown) into a body lumen of a patient and moving elongate shaft 108 and neuromodulation element 112 along the guidewire until neuromodulation element 112 reaches a suitable treatment location. Alternatively, neuromodulation catheter 102 may be a steerable or non-steerable device configured for use without a guidewire. Additionally, or alternatively, neuromodulation catheter 102 may be configured for use with another type of guide member, such as a guide catheter or a sheath (not shown), alone, or in addition to a guidewire.

RF generator 104 is configured to control, monitor, supply, and/or otherwise support operation of neuromodulation catheter 102. In other examples, neuromodulation catheter 102 may be self-contained or otherwise configured for operation independent of RF generator 104. When present, RF generator 104 is configured to generate a selected form and/or magnitude of RF energy for delivery to tissue at a treatment location via neuromodulation element 112. For example, RF generator 104 may be configured to generate RF energy (e.g., monopolar and/or bipolar RF energy). In other examples, RF generator 104 may be another type of device configured to generate and deliver another suitable type of energy to neuromodulation element 112 for delivery to tissue at a treatment location via neuromodulation element 112.

Along cable 106 or at another suitable location within therapeutic system 100, therapeutic system 100 may include a control device 114 configured to initiate, terminate, and/or adjust operation of one or more components of neuromodulation catheter 102 directly and/or via RF generator 104. RF generator 104 may be configured to execute an automated control algorithm 116 and/or to receive control instructions from an operator. Similarly, in some examples, RF generator 104 is configured to provide feedback to an operator before, during, and/or after a treatment procedure via an evaluation/feedback algorithm 118.

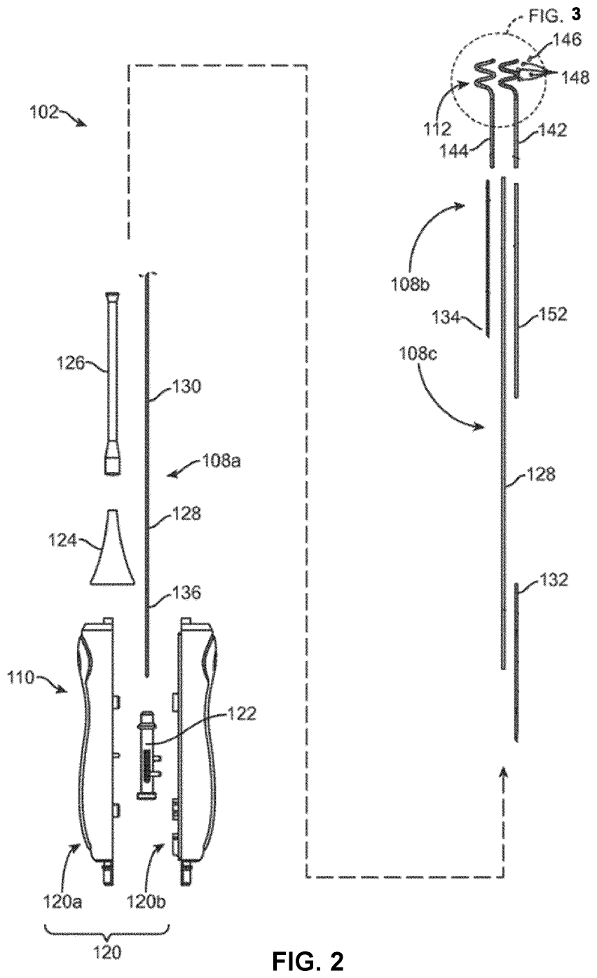

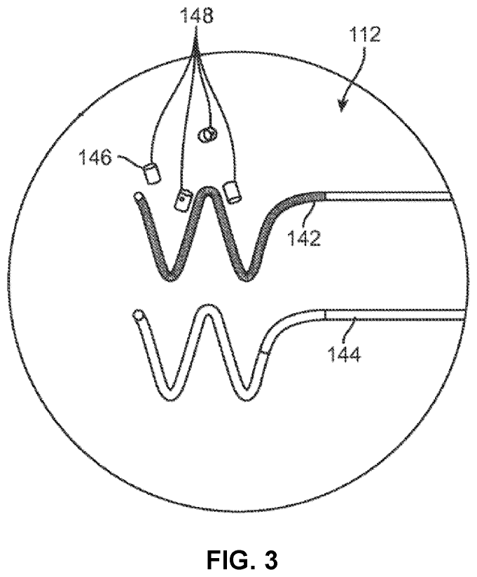

FIG. 2 is an exploded profile view of neuromodulation catheter 102. FIG. 3 is an enlarged exploded profile view of portions of neuromodulation catheter 102 taken at the location designated in FIG. 2. With reference to FIG. 2, handle 110 may include mating shell segments 120 (individually identified as shell segments 120a, 120b) and a connector 122 (e.g., a luer connector) operably positioned between the mating shell segments 120. Handle 110 may further include a distally tapered strain-relief element 124 operably connected to distal ends of shell segments 120. Slidably positioned over elongate shaft 108, neuromodulation catheter 102 may include a loading tool 126 configured to facilitate loading catheter 102 onto a guidewire (not shown). When assembled, elongate shaft 108 may extend through coaxial lumens (also not shown) of strain-relief element 124 and loading tool 126, respectively, and between shell segments 120 to connector 122.

Elongate shaft 108 may include an assembly of parallel tubular segments. At proximal portion 108a and extending distally though a majority of intermediate portion 108c, elongate shaft 108 may include a metal hypotube 128, a proximal outer jacket 130, and a first electrically insulative tube 132. Proximal outer jacket 130 may be a polymer outer jacket disposed around at least a portion of an outer surface of metal hypotube 128. First electrically insulative tube 132 may be an electrically insulative polymer tube or coating disposed within an inner lumen of metal hypotube 128. First electrically insulative tube 132 may define an inner lumen, and one or more electrical leads (e.g., wires 148) may be positioned within the inner lumen of first electrically insulative tube 132. First electrically insulative tube 132 may electrically insulate the electrical leads from metal hypotube 128.

Elongate shaft 108 may include a single metal hypotube 128. Metal hypotube 128 may be configured to bend with varying flexibility along a length of metal hypotube 128. For example, in proximal portion 108a, metal hypotube 128 may be configured to bend with a first flexibility along the length of proximal portion 108a. In intermediate portion 108c, metal hypotube 128 may be configured to bend with a second flexibility along the length of intermediate portion 108c, and in distal portion 108b, metal hypotube 128 may be configured to bend with a third flexibility along the length of distal portion 108b. The first, second, and third flexibilities may all be different from one another. In some examples, metal hypotube 128 is configured to bend with greater flexibility in a distal portion of metal hypotube 128 than a proximal portion, such that each transition in flexibility from a proximal end of metal hypotube 128 to a distal end of metal hypotube 128 is a transition from a less flexible portion of metal hypotube 128 to a more flexible portion. For example, the first flexibility may be less than or equal to the second flexibility, which may be less than or equal to the third flexibility, such that the intermediate portion is more flexible than the portion proximal, and the distal portion is more flexible than the intermediate portion.

In some examples, the flexibility of a portion of metal hypotube 128 may be configured to allow that portion of metal hypotube 128 to bend with a bending radius. For example, proximal portion 108a of metal hypotube 128 may have a first flexibility configured to allow proximal portion 108a to bend with a first bending radius, intermediate portion 108c may have a second flexibility configured to allow intermediate portion 108c to bend with a second bending radius, and distal portion 108b may have a third flexibility configured to allow distal portion 108b to bend with a third bending radius. Each portion of metal hypotube 128 may be configured to bend with a smaller bending radius than the portion of metal hypotube 128 proximal to it. For example, the second bending radius of intermediate portion 108c may be smaller than the first bending radius of proximal portion 108a, and the third bending radius of distal portion 108b may be smaller than the second bending radius of intermediate portion 108c. In other examples, the flexibility or bending radius may change gradually or continuously along a length or a portion of a length of metal hypotube 128 (e.g., as compared to step-wise). Metal hypotube 128 may be made at least partially of Nitinol, stainless steel, or another suitable metal.

Metal hypotube 128 may define a rapid exchange joint opening (not shown) in intermediate portion 108c of metal hypotube 128. The rapid exchange joint opening is an opening in a sidewall of metal hypotube sized and shaped to enable a guidewire tube to extend into and/or through the rapid exchange joint opening to allow a guidewire to transition from within the elongate body 108 to outside elongate body 108. Metal hypotube 128 may include a distal end, a proximal end, and a length therebetween. In some examples, the rapid exchange joint opening is positioned along the length of metal hypotube 128 between sixty and ninety percent of the length, as measured from the proximal end of metal hypotube 128.

With reference again to FIGS. 2 and 3, the first electrically insulative tube 132 may extend distally from proximal portion 108a into intermediate portion 108c. The portion of metal hypotube 128 in intermediate portion 108c may be more flexible than the portion of metal hypotube 128 in proximal portion 108a. At or near the region of elongate shaft 108 where metal hypotube 128 defines a rapid exchange joint opening, first electrically insulative tube 132 may terminate at a distal end of first electrically insulative tube 132.

A proximal portion of guidewire tube 134 may be positioned at least partially in the rapid exchange joint opening to allow a guidewire to pass from the inside of metal hypotube 128 distal to the rapid exchange joint opening to the outside of the catheter proximal of the rapid exchange joint opening. The proximal portion of guidewire tube 134, including a proximal end of guidewire tube 134, may be attached to metal hypotube 128 (e.g., using a reflowed polymer, an adhesive, or the like). The proximal portion of guidewire tube 134 may include a skived section defining a smooth diameter transition between proximal outer jacket 130 (i.e., a polymer outer jacket) and a location where the proximal portion of guidewire tube 134 extends into the rapid exchange joint opening. In some examples, the proximal end of guidewire tube 134 may terminate outside of metal hypotube 128 proximal of the rapid exchange joint opening. In some examples, the proximal end of guidewire tube 134 may terminate at the exchange joint opening. The guidewire may be positioned inside an interior lumen of guidewire tube 134 in a distal portion 108b of metal hypotube 128, exit metal hypotube 128 at the rapid exchange joint opening, and be positioned outside the catheter from the rapid exchange joint opening proximally of the rapid exchange joint opening (e.g., to at least handle 110). Guidewire tube 134 may extend distally from the rapid exchange joint opening to neuromodulation element 112. At a distal end of metal hypotube 128, elongate shaft 108 may be operably connected to the neuromodulation element 112.

In FIGS. 2 and 3, neuromodulation element 112 is shown in a radially expanded deployed state. Neuromodulation element 112 may be movable from a low-profile delivery state to the radially expanded deployed state. When neuromodulation element 112 is in the radially expanded deployed state, a distal shape-memory structure 142, such as a Nitinol-based structure (such as a helical hollow strand (HHS®) structure, available from Fort Wayne Metals, Fort Wayne, Indiana), or another shape-memory material, may have a shape that is more helical (spiral) than its shape when neuromodulation element 112 is in the low-profile delivery state. In at least some cases, distal shape-memory structure 142 has the more helical shape when at rest and is configured to be urged into the less helical shape by an external sheath (not shown), an internal guidewire, an internal mandrel, or the like. In at least some cases, the material of distal shape-memory structure 142 is electrically conductive. Accordingly, neuromodulation element 112 may include a second electrically insulative tube 152 disposed around an outer surface of distal shape-memory structure 142 so as to electrically separate electrodes 146 from distal shape-memory structure 142. In some examples, first and second electrically insulative tubes 132, 152 are made at least partially (e.g., predominantly or entirely) of polyimide and polyether block amide, respectively. In other examples, first and second electrically insulative tubes 132, 152 may be made of other suitable materials, e.g., other suitable polymers. Distal jacket 144 may be tubular and configured to be disposed around at least a portion of an outer surface of distal shape-memory structure 142.

Neuromodulation element 112 may include one or more electrodes 146 (e.g., one electrode, two electrodes, three electrodes, four electrodes, or the like) and may be configured to transform between a substantially straight delivery configuration and a deployed configuration (e.g., a spiral or helical configuration). Neuromodulation catheter 102 may further include one or more wires 148 or wire pairs extending from a proximal end (or from near the proximal end) of neuromodulation catheter 102 to the one or more electrode(s) 146 at distal portion 108b of neuromodulation catheter 102, each wire (or wire pair) of one or more wires 148 being electrically coupled (e.g., welded or otherwise affixed) to a corresponding electrode of one or more electrodes 146 to deliver energy, and in some examples, to form a thermocouple for conducting temperature measurements. Distal portion 108b of neuromodulation catheter 102 may also define one or more openings or slots through which the one or more wire(s) 148 extend in order to contact and electrically couple to the one or more electrode(s) 146.

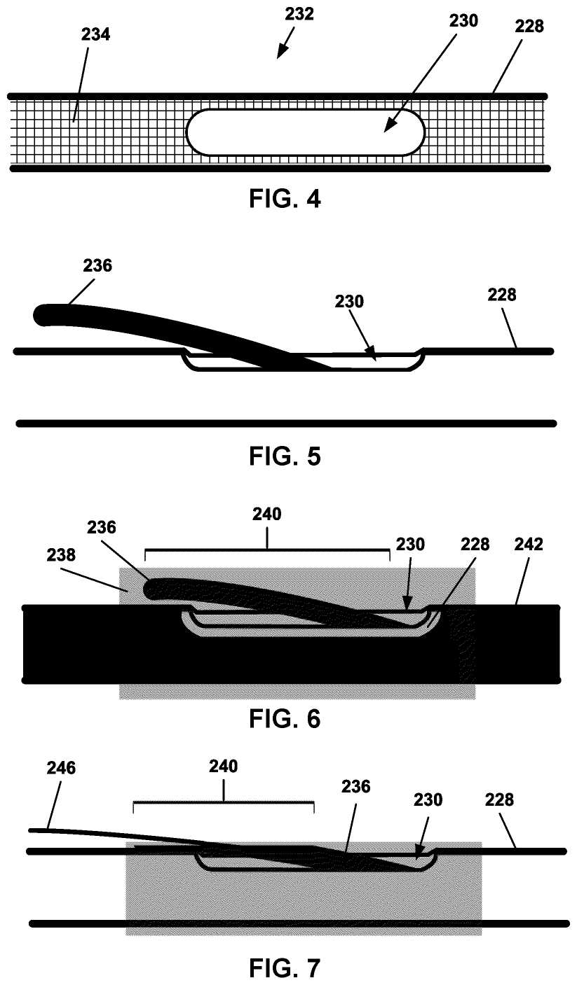

FIGS. 4-7 are images of an example intermediate portion of a metal hypotube in various states of assembly with a guidewire tube. FIG. 4 is a side view of an example metal hypotube 228 with a rapid exchange joint opening 230, in accordance with one or more examples of the present disclosure. Rapid exchange joint opening 230 may be located in an intermediate portion 232 of metal hypotube 228. Metal hypotube 228 may be configured to bend to navigate tortious anatomy of a patient. For example, metal hypotube 228 may include a plurality of holes 234 defined in a tube wall of metal hypotube 228. Plurality of holes 234 may be formed in one or more patterns, and the one or more patterns may be selected to achieve a desired flexibility for that portion of metal hypotube 228. For example, the pattern of plurality of holes 234 may include a size of holes 234, a shape of plurality of holes 234, a spacing (or alternatively, areal density) of plurality of holes 234, or the like. As an example, the less metal remaining within the pattern, the more flexible that section of metal hypotube 228 may be. In some examples, plurality of holes 234 may formed using a laser cutting process (e.g., plurality of holes 234 may be laser-cut holes).

Rapid exchange joint opening 230 may be cut, ablated, machined, or otherwise removed from a tube wall of intermediate portion 232 of metal hypotube 228. A size and shape of rapid exchange joint opening 230 may be selected to allow a guidewire tube to extend into and/or through rapid exchange joint opening 230 while having a guidewire lumen within the guidewire tube sufficiently large to accommodate a guidewire. Conversely, the size and shape of rapid exchange joint opening 230 may be selected such that rapid exchange joint opening 230 does not compromise mechanical properties of metal hypotube 228, such as causing metal hypotube 228 to be more likely to fold or buckle at or near rapid exchange joint opening 230. In some examples, rapid exchange joint opening 230 may be formed with smooth edges to avoid catching on patient anatomy, or cutting against internal components of the catheter.

FIG. 5 is a side view of example metal hypotube 228 with a guidewire tube 236 passing through rapid exchange joint opening 230, in accordance with one or more examples of the present disclosure. A proximal portion of guidewire tube 236 may be positioned at least partially in rapid exchange joint opening 230 to allow a guidewire to pass from the inside of metal hypotube 228 distal to rapid exchange joint opening 230 to the outside of the catheter proximal of rapid exchange joint opening 230.

FIG. 6 is a side view of an example metal hypotube 228 and guidewire tube 236 connected by reflowed polymer 238, in accordance with one or more examples of the present disclosure. Proximal portion 240 of guidewire tube 236 may be attached to metal hypotube 228 by reflowed polymer 238. Reflowed polymer 238 may secure proximal portion 240 of guidewire tube 236 to metal hypotube 228 in the area of the rapid exchange joint opening 230. Reflowed polymer 238 may also serve to smooth any edges of guidewire tube 236 and rapid exchange joint opening 230. In some examples, reflowed polymer 238 may be made using heat shrink tubing, for example with an expanded inner diameter of around 0.060 inches and a recovered inner diameter of around 0.040 inches.

In some examples, metal hypotube 228 includes an external coating or layer 242 (also referred to as a polymer outer jacket 242). For example, metal hypotube 228 may include a polymer coating or layer 242 that is on an outer surface of metal hypotube 228. The polymer coating or layer 242 may include any biocompatible polymer or mixture of polymers, such as silicone, a polyurethane, a polyimide, a polyether block amide, or the like. In some examples, external coating or layer 242 may be an example of proximal outer jacket 130 of FIGS. 1 and 2. Reflowed polymer 238 may bond guidewire tube 236 to polymer coating or layer 242. Reflowed polymer 238 may include a separate portion of material from polymer coating or layer 242 and from guidewire tube 236. Reflowed polymer 238 may include any biocompatible polymer or mixture of polymers, such as silicone, a polyurethane, a polyimide, a polyether block amide, or the like. In some examples, reflowed polymer 238 may include a polyether block amide, such as a PEBAX®, available from Arkema, Inc., The King of Prussia, Pennsylvania. The PEBAX® may in some examples be mixed with a second polymer, such as a siloxane. In other examples, guidewire tube 236 may be attached to metal hypotube 228 using another technique, such as an adhesive.

Once attached to metal hypotube 228 (e.g., using a reflowed polymer 238), guidewire tube 134 may be cut to include a skived section 244, as shown in FIG. 7. Skived section 244 may define a smooth diameter transition between external coating or layer 242 and a location where a portion of the guidewire tube 236 extends into rapid exchange joint opening 230. In some examples, a cross section of metal hypotube 228 with external coating or layer 242 may define a circle, an ellipse, an oval, or the like. As guidewire tube 236 exits metal hypotube 228 through rapid exchange joint opening 230, the added width of guidewire tube 236 may create a bump on the outer surface of metal hypotube 228. The bump increases an effective diameter of the catheter, and may define an unwanted protrusion. Skived section 244 of guidewire tube 136 substantially removes any bump or protrusion by bringing a cross sectional profile of guidewire tube 236 and reflowed polymer 238 to substantially match an outer diameter of metal hypotube 228 and external coating or layer 242.

Guidewire 246 may be positioned inside guidewire tube 236 in a section of the catheter distal to rapid exchange joint opening 230. Guidewire 246 may exit metal hypotube 228 and guidewire tube 236 at rapid exchange joint opening 230 and be positioned outside metal hypotube 228 and external coating or layer 242 (e.g., outside the catheter) in a section of the catheter proximal to rapid exchange joint opening 230.

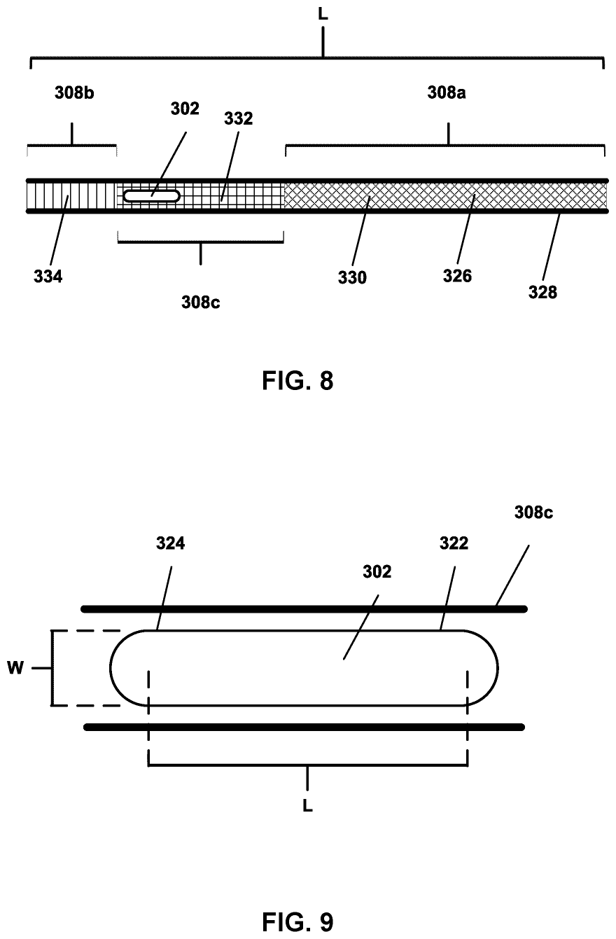

FIG. 8 is a side view of an example metal hypotube 328 with varying flexibility along a length, L, of metal hypotube 328, in accordance with one or more examples of the present disclosure. Metal hypotube 328 may be an example of metal hypotubes 128 or 228 of FIGS. 1-7. Metal hypotube 328 may include a proximal portion 308a configured to bend with a first flexibility along a length of proximal portion 308a, an intermediate portion 308c configured to bend with a second flexibility along a length of the intermediate portion 308c, and a distal portion 308b configured to bend with a third flexibility along a length of distal portion 308b. The first, second, and third flexibilities may all differ from one another. By having different sections of metal hypotube 328 with different flexibilities, metal hypotube 328 may have varying flexibility along its length, L. In some examples, the flexibility increases along the length, L, from a proximal end of metal hypotube 328 to a distal end of metal hypotube 328. For example, intermediate portion 308cmay be more flexible than proximal portion 308a, and distal portion 308b may be more flexible than intermediate portion 308c.

In some examples, the change in flexibility may be a step change (e.g., an abrupt change from one value to another) at a border between proximal portion 308a and intermediate portion 308c and a border between intermediate portion 308c and distal portion 308b. In other examples, the change in flexibility may be gradual (e.g., continuous or semi-continuous) along a portion of a length of metal hypotube 328. For example, the change in flexibility may be gradual within intermediate portion 308c, within distal portion 308b, and/or within proximal portion 308a. In some examples, the second flexibility along the length of intermediate portion 308c gradually increases along the length of intermediate portion 308c from a proximal end of intermediate portion 308c to a distal end of intermediate portion 308c.

In some examples, the flexibility of a portion of metal hypotube 328 may be configured to allow that portion of metal hypotube 328 to bend with a bending radius. A larger bending radius may be associated with a lower flexibility. Proximal portion 308a of metal hypotube 328 may have a first flexibility configured to allow proximal portion 308a to bend with a first bending radius, intermediate portion 308c may have a second flexibility configured to allow intermediate portion 308c to bend with a second bending radius, and distal portion 308b may have a third flexibility configured to allow distal portion 308b to bend with a third bending radius. The second bending radius may be smaller than the first bending radius, and the third bending radius may be smaller than the second bending radius. For example, the first flexibility may allow proximal portion 308a to bend with a bending radius of about two inches, the second flexibility may allow intermediate portion 308c to bend with a bending radius of about one point five inches, and the third flexibility may allow distal portion 308b to bend with a bending radius of about one inch. In some examples, the second bending radius may gradually change along a length of intermediate portion 308c. For example, the second bending radius may gradually change from a two-inch bending radius at a proximal end of intermediate portion 308c to a one-inch bending radius at a distal end of intermediate portion 308c.

Rapid exchange joint opening 302 may be positioned along length L of metal hypotube 328 at any selected location. In some examples, rapid exchange joint opening 302 is positioned within intermediate portion 308c. In some examples, rapid exchange joint opening 302 may be positioned at a position that is between about sixty percent of the length L of metal hypotube 328 from a proximal end of metal hypotube 328 and about ninety percent of the length L of metal hypotube 328 from a proximal end of metal hypotube 328.

Metal hypotube 328 may include a tube wall 326. Tube wall 326 includes a plurality of holes in one or more patterns. The plurality of holes are configured to allow metal hypotube 328 to bend with a selected flexibility. The portion of tube wall 326 in proximal portion 308a of metal hypotube 328 may include a plurality of holes in a first pattern 330 configured to allow proximal portion 308a to bend with the first flexibility. The portion of tube wall 326 in intermediate portion 308c of metal hypotube 328 may include a plurality of holes in a second pattern 332 configured to allow intermediate portion 308c to bend with the second flexibility. In some examples, rapid exchange joint opening 302 may be sized, and the second pattern 332 may be configured such that that rapid exchange joint opening 302 does not compromise the flexibility of intermediate portion 308c at rapid exchange joint opening 302. The portion of tube wall 328 in distal portion 308b of metal hypotube 328 may include a plurality of holes in a third pattern 334 configured to allow distal portion 308b to bend with the third flexibility. The patterns depicted in FIG. 8 are depicted for reference only, and should not be limited based on their appearance in FIG. 8.

The pattern(s) of the plurality of holes may include a size of the holes, a shape of the holes, a spacing (or alternatively, areal density) of the holes, or the like. As an example, the less metal remaining within the pattern, the more flexible that section of metal hypotube 328 may be. In some examples, the plurality of holes may be formed using a laser cutting process (e.g., the plurality of holes may be laser-cut holes). In this way, by having a single hypotube having a varying flexibility along its length, metal hypotube 328 may serve the function of effectively navigating patient anatomy, while saving time during assembly of the catheter by reducing a number of components that need to be assembled to form the catheter.

FIG. 9 is an enlarged view of an example rapid exchange joint opening 302 in intermediate portion 308c of the metal hypotube 328 of FIG. 8, in accordance with one or more examples of the present disclosure. Rapid exchange joint opening 302 may allow a guidewire and guidewire tube to pass from the inside of metal hypotube 328 distal to rapid exchange joint opening 302 to the outside of metal hypotube 328 proximal of rapid exchange joint opening 302.

In some examples, rapid exchange joint opening 302 has a length L that is as small as possible while allowing the guidewire tube to exit metal hypotube 328. In some examples, rapid exchange joint opening 302 has a length L that is significantly larger than a diameter of the guidewire tube to allow the guidewire tube to exit the metal hypotube 328 at a shallow angle. The shallow angle may provide a smoother transition between an outer diameter of metal hypotube 328 proximal to rapid exchange joint opening 302, and an outer diameter of metal hypotube 328 and exiting guidewire tube at rapid exchange joint opening 302. The shallow angle may also prevent a guidewire from getting caught at rapid exchange joint opening 302. The guidewire may be positioned inside a lumen of the guidewire tube inside a lumen of metal hypotube 328 in a portion of the catheter distal to rapid exchange joint opening 302, and may be positioned outside metal hypotube 328 for a portion of the catheter proximal to rapid exchange joint opening 302.

In some examples, a distal portion of rapid exchange joint opening 302 and a proximal portion of rapid exchange joint opening 302 may each define a radius of curvature. In some examples, a length L of rapid exchange joint opening 302 is measured from the center of the radius of curvature of the proximal portion of rapid exchange joint opening 302 to the center of the radius of curvature of the distal portion of rapid exchange joint opening 302. In other examples, length L of rapid exchange joint opening 302 is measured from a distal end of rapid exchange joint opening 302 to a proximal end of rapid exchange joint opening 302, as with calipers. In some examples, length L is between about 0.15 inches and about 0.3 inches, such as between about 0.18 inches and about 0.25 inches. For example, length L may be about 0.18 inches, or about 0.20 inches, or about 0.22 inches, or about 0.25 inches.

In some examples, rapid exchange joint opening 302 has a width W that is as small as possible while allowing the guidewire tube to exit metal hypotube 328. In other examples, rapid exchange joint opening 302 has width W that is significantly larger than a diameter of the guidewire tube. In these examples, assembly of the catheter may be made easier, as there may not be as precise a fit between the guidewire tube and rapid exchange joint opening 302. Rapid exchange joint opening 302 may be sized such that rapid exchange joint opening 302 does not compromise the structural integrity of the catheter at rapid exchange joint opening 302. In some examples, rapid exchange joint opening 302 has width W that is no greater than eighty percent of an inner diameter of the metal hypotube and no less than an outer diameter of the guidewire tube. In some examples, width W is between about 0.02 inches and about 0.04, such as between about 0.024 inches and about 0.030 inches. In some examples, width W is about 0.024 inches, about 0.028 inches, about 0.029 inches, or about 0.030 inches.

In some examples, rapid exchange joint opening 302 may be tapered such that width W differs on a proximal portion 322 of rapid exchange joint opening 302 from a distal portion 324 of rapid exchange joint opening 302. For example, width W may be larger on proximal portion 322 of rapid exchange joint opening 302 than distal portion 324 of rapid exchange joint opening 302. In some examples, width W may be larger on distal portion 324 of rapid exchange joint opening 302 than proximal portion 322of rapid exchange joint opening 302. For example, width W may be about 0.29 inches on proximal portion 322 of rapid exchange joint opening 302 and about 0.24 inches on distal portion 324 of rapid exchange joint opening 302, or width W may be about 0.29 inches on distal portion 324 of rapid exchange joint opening 302 and about 0.24 inches on proximal portion 322 of rapid exchange joint opening 302.

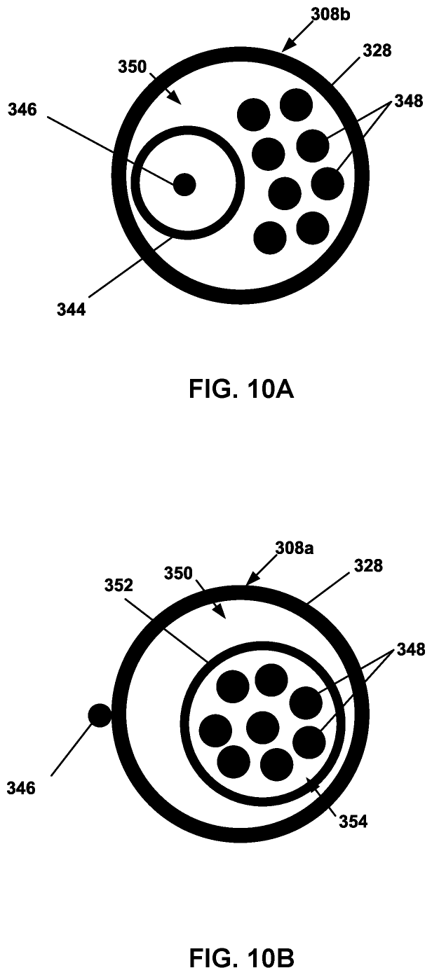

FIG. 10A is a cross sectional view of a distal portion 308b of metal hypotube 328 and other components of a catheter, where distal portion 308b is distal to rapid exchange joint opening 302. The catheter may include one or more wires 348 (i.e., electrical leads) or wire pairs extending from a proximal end (or from near the proximal end) of the catheter to the electrode(s) at the distal portion of the catheter. One or more wires 348 may include one or more wire for each electrode at the distal portion of the catheter. For example, one or more wires 348 may include a first wire and a second wire for each electrode, such that the catheter includes twice the number of wires as electrodes. As another example, one or more wires 348 may include a single wire for each electrode, such that the catheter includes the same number of wires as electrodes. In examples in which one or more wires 348 may include a first wire and a second wire for each electrode, each wire or wire pair may be electrically coupled to a corresponding electrode to deliver energy and act as a thermocouple for sensing temperature at the electrode. In distal portion 308b of metal hypotube 328, a guidewire tube 344 tube may be positioned within a lumen 350 of metal hypotube 328. A guidewire 346 may be positioned inside a guidewire lumen of guidewire tube 344. Furthermore, in distal portion 308b of metal hypotube 328, wires 348 may be positioned within lumen 350 of metal hypotube 328 alongside (e.g., substantially parallel to and outside of guidewire tube 344). In some examples, wires 348 may be positioned inside a second electrically insulative tube as described with reference to FIGS. 2 and 3 in at least a portion of distal portion 308b.

FIG. 10B is a cross sectional view of a proximal portion 308a of metal hypotube 328, where proximal portion 308a is proximal to a rapid exchange joint opening 302. In proximal portion 308a of metal hypotube 328, the one or more wires 348 (e.g., electrical leads) may be positioned inside a first electrically insulative tube 352. First electrically insulative tube 352 may be an electrically insulative polymer disposed within lumen 350 of metal hypotube 328. In other examples, rather than being a separate tube, first electrically insulative tube 352 may instead be a coating or layer on an inner surface of metal hypotube 328. First electrically insulative tube 352 may define a lumen 354, wherein one or more electrical leads (e.g., wires 348) are positioned within lumen 354 of first electrically insulative tube 352. First electrically insulative tube 352 may electrically insulate one or more wires 348 from the metal hypotube 328. Guidewire 346 may be positioned on the outside of metal hypotube 328 proximal to the rapid exchange joint opening 302, as guidewire 346 may be extend through the rapid exchange joint opening 302.

The sizes of wires 348 and guidewire 346 in FIGS. 10A and 10B are depicted for ease of visibility only, and should not be limited to their size with respect to the portions 308a and 308b of metal hypotube 302 depicted in FIGS. 10A and 10B.

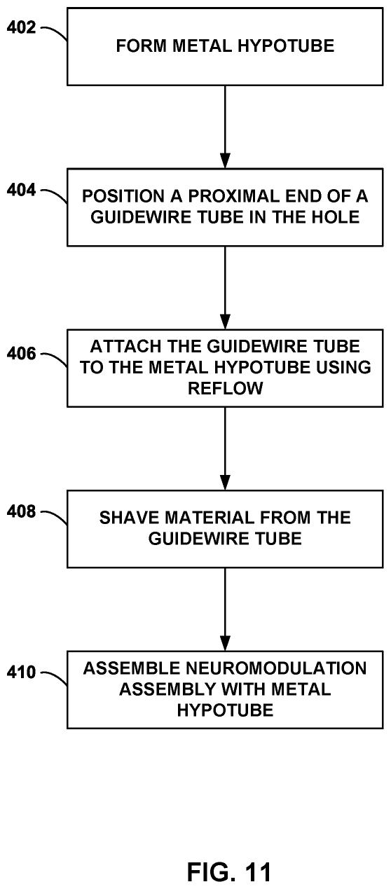

FIG. 11 is a flow chart illustrating a method 600 for manufacturing a neuromodulation catheter, in accordance with one or more examples of the present disclosure. The technique of FIG. 11 will be described with concurrent reference to FIGS. 4-7, 10A, and 10B for ease of description. A person having ordinary skill in the art will appreciate that the technique of FIG. 11 may be performed using other components, and that the components of FIGS. 4-7, 10A, and 10B may be used in other assembly techniques. For example, the technique of FIG. 11 may optionally include forming metal hypotube 228 (402). In other examples, the technique of FIG. 11 may include obtaining a metal hypotube 228 formed as described herein.

As described above, metal hypotube 228 may include a rapid exchange joint opening 230 and a plurality of holes 234. Forming metal hypotube 228 may include cutting rapid exchange joint opening 230 and forming plurality of holes 234 in metal hypotube 228. For example, rapid exchange joint opening 230 and plurality of holes 234 may be laser cut in metal hypotube 228.

In some examples, forming metal hypotube 228 (402) may include laser-cutting plurality of holes 234 in one or more patterns in a tube wall of metal hypotube 228. The plurality of holes 234 in one or more patterns are configured to allow the metal hypotube to bend with varying flexibility along a length of metal hypotube 228. For example, forming metal hypotube (402) may include laser-cutting a plurality of holes 234 in the tube wall of a proximal portion of metal hypotube 228 in a first pattern configured to allow the proximal portion of metal hypotube 228 to bend with a first flexibility. The first flexibility may be a relatively lower flexibility. In some examples, forming metal hypotube (402) may include laser-cutting a plurality of holes 234 in the tube wall of an intermediate portion of metal hypotube 228 in a second pattern configured to allow the intermediate portion of metal hypotube 228 to bend with a second flexibility. The second flexibility may greater than the first flexibility. In some examples, forming metal hypotube (402) may include laser-cutting a plurality of holes 234 in the tube wall of a distal portion of metal hypotube 228 in a third pattern configured to allow the distal portion of metal hypotube 228 to bend with a third flexibility. In some examples, the third flexibility is greater than the second flexibility and greater than the first flexibility.

In some examples, forming metal hypotube (402) includes laser-cutting plurality of holes 234 in the tube wall of one or more portions of metal hypotube 228 in a pattern configured to provide a gradually changing flexibility along the length of the one or more portions. In some examples, the flexibility of a portion of metal hypotube 228may allow that portion of metal hypotube 228 to bend with a bending radius. The proximal portion of metal hypotube 228 may have a first flexibility configured to allow the proximal portion to bend with a first bending radius, the intermediate portion of metal hypotube 228 may have a second flexibility configured to allow the intermediate portion to bend with a second bending radius, and the distal portion of metal hypotube 228 may have a third flexibility configured to allow the distal portion to bend with a third bending radius. The second bending radius may be smaller than the first bending radius, and the third bending radius may be smaller than the second bending radius. A larger bending radius may be associated with a lower flexibility. In some examples, the second flexibility may gradually increase along the length of the intermediate portion from a proximal end of the intermediate portion to a distal end of the intermediate portion. For example, the second bending radius of the intermediate portion may gradually change from a two-inch bending radius at a proximal end of the intermediate portion to a one-inch bending radius at a distal end of the intermediate portion.

Forming metal hypotube (402) also may include cutting rapid exchange joint opening 230 in intermediate portion 232 of metal hypotube. Cutting rapid exchange joint opening 230 may include ablating, machining, or otherwise removing material from a tube wall of intermediate portion 232 of metal hypotube 228 to form rapid exchange joint opening 230. A size and shape of rapid exchange joint opening 230 may be selected to allow a guidewire tube to extend into and/or through rapid exchange joint opening 230 while having a guidewire lumen within the guidewire tube sufficiently large to accommodate a guidewire. Conversely, the size and shape of rapid exchange joint opening 230 may be selected such that rapid exchange joint opening 230 does not compromise mechanical properties of metal hypotube 228, such as causing metal hypotube 228 to be more likely to fold or buckle at or near rapid exchange joint opening 230. In some examples, cutting rapid exchange joint opening 230 may include forming rapid exchange joint opening 230 with smooth edges to avoid catching on patient anatomy, or cutting against internal components of the catheter.

In some examples, the metal hypotube may include a distal end, a proximal end, and a length therebetween, and cutting rapid exchange joint opening 230 may include cutting rapid exchange joint opening 230 at a position along the length of metal hypotube 228 between sixty percent and ninety percent of the length from the proximal end to the distal end.

In some examples, cutting rapid exchange joint opening 230 may include cutting rapid exchange joint opening 230 with a length L (e.g., length L of FIG. 9) that is as small as possible while allowing the guidewire tube 236 to exit metal hypotube 228. In some examples, rapid exchange joint opening 230 has a length L that is significantly larger than a diameter of the guidewire tube 236 to allow the guidewire tube 236 to exit the metal hypotube 228 at a shallow angle. The shallow angle may provide a smoother transition between an outer diameter of metal hypotube 228 proximal to rapid exchange joint opening 230, and an outer diameter of metal hypotube 228 and exiting guidewire tube 236 at rapid exchange joint opening 230. The shallow angle may also prevent a guidewire 246 from getting caught at rapid exchange joint opening 230.

In some examples, a distal portion of rapid exchange joint opening 230 and a proximal portion of rapid exchange joint opening 230 may each define a radius of curvature after cutting rapid exchange joint opening 230 in metal hypotube 228. In some examples, a length L of rapid exchange joint opening 230 is measured from the center of the radius of curvature of the proximal portion of rapid exchange joint opening 230 to the center of the radius of curvature of the distal portion of rapid exchange joint opening 230. In other examples, length L of rapid exchange joint opening 230 is measured from a distal end of rapid exchange joint opening 230 to a proximal end of rapid exchange joint opening 230, as with calipers. In some examples, cutting rapid exchange joint opening 230 in metal hypotube 228 includes forming the length L between about 0.15 inches and about 0.3 inches, such as between about 0.18 inches and about 0.25 inches. For example, forming length L about 0.18 inches, or about 0.20 inches, or about 0.22 inches, or about 0.25 inches.

In some examples, cutting rapid exchange joint opening 230 may include forming a width W (e.g., width W of FIG. 9) of rapid exchange joint opening 230 that is as small as possible while allowing the guidewire tube to exit metal hypotube 228. In these examples, rapid exchange joint opening 230 may have the smallest possible effect on a structural integrity of metal hypotube 228. In other examples, cutting rapid exchange joint opening 230 may include forming width W that is significantly larger than a diameter of the guidewire tube. In these examples, assembly of the catheter may be made easier, as there may not be as precise a fit between the guidewire tube and rapid exchange joint opening 230. Rapid exchange joint opening 302 may be sized such that rapid exchange joint opening 230 does not compromise the structural integrity of the catheter at rapid exchange joint opening 230. In some examples, cutting rapid exchange joint opening 230 includes cutting rapid exchange joint opening 230 with width W no greater than eighty percent of an inner diameter of the metal hypotube and no less than an outer diameter of the guidewire tube. In some examples, width W may be between about 0.02 inches and about 0.04, such as between about 0.024 inches and about 0.030 inches. In some examples, width W may be about 0.024 inches, about 0.028 inches, about 0.029 inches, or about 0.030 inches.

In some examples, cutting rapid exchange joint opening 230 includes cutting rapid exchange joint opening 230 with a taper such that width W differs on a proximal portion of rapid exchange joint opening 230 from a distal portion of rapid exchange joint opening 230. For example, width W may be larger on the proximal portion of rapid exchange joint opening 230 than the distal portion of rapid exchange joint opening 230. In some examples, width W may be larger on the distal portion of rapid exchange joint opening 230 than the proximal portion of rapid exchange joint opening 230. For example, width W may be about 0.29 inches on a proximal portion of rapid exchange joint opening 230 and about 0.24 inches on a distal portion of rapid exchange joint opening 230, or width W may be about 0.29 inches on a distal portion of rapid exchange joint opening 230 and about 0.24 inches on a proximal portion of rapid exchange joint opening 230.

The technique of FIG. 11 may further include positioning a proximal portion 240 of guidewire tube 236 at least partially in rapid exchange joint opening 230 to enable a guidewire 246 to pass from a lumen of metal hypotube 228 distal of rapid exchange joint opening 230 to the outside of the catheter proximal of rapid exchange joint opening 230 (404). In some examples, proximal portion 240 of guidewire tube 236 may be positioned at least partially in rapid exchange joint opening 230 by pulling a proximal end of guidewire tube 236 proximally through the lumen of metal hypotube 228 from a distal end of metal hypotube 228 using a mandrel or other tool. In some examples, proximal portion 240 of guidewire tube 236 may be positioned at least partially in rapid exchange joint opening 230 by feeding a distal end of guidewire tube 236 through rapid exchange joint opening 230 and distally through the lumen of metal hypotube 228 until proximal portion 240 of guidewire tube 236 is positioned at least partially in rapid exchange joint opening 230.

The technique of FIG. 11 may further include attaching guidewire tube 236 to metal hypotube 228 (406). Attaching guidewire tube 236 to metal hypotube 228 may include using reflowed polymer 238, an adhesive, or other method of attaching guidewire tube 236 and metal hypotube 228. Guidewire tube 236 may be fixed at proximal portion 240 of guidewire tube 236 to metal hypotube 282 at or just proximal to rapid exchange joint opening 230. Reflowed polymer 238 may also smooth any edges of a proximal portion 240 of guidewire tube 236 and any edges of rapid exchange joint opening 230. Metal hypotube 228 may include a polymer coating or layer 242 that is on an outer surface of metal hypotube 228. The polymer coating or layer 242 may include any biocompatible polymer or mixture of polymers, such as silicone, a polyurethane, a polyimide, a polyether block amide, or the like. Attaching guidewire tube 236 to metal hypotube 228 (406) may include using reflowed polymer 238 to bond guidewire tube 236 to polymer coating and/or layer 242 simultaneously. For example, reflowed polymer 238 and layer 242 may be heated to 350 degrees Fahrenheit in a wider nozzle hot box. Reflowed polymer 238 may include a separate portion of material from polymer coating or layer 242 and from guidewire tube 236. Reflowed polymer 238 may include any biocompatible polymer or mixture of polymers, such as silicone, a polyurethane, a polyimide, a polyether block amide, or the like.

In some examples, a cross section of metal hypotube 228 with external coating or layer 242 may define a circle, an ellipse, an oval, or the like. As guidewire tube 236 exits metal hypotube 228 through rapid exchange joint opening 230, the added width of guidewire tube 236 may create a bump on the outer surface of metal hypotube 228. The bump increases an effective diameter of the catheter, and may define an unwanted protrusion.

The technique of FIG. 11 may further include shaving material from guidewire tube 236 to create a substantially smooth circumferential transition between a polymer outer jacket over metal hypotube 228 and a location where a portion of guidewire tube 236 extends outward from metal hypotube 228 (408). Shaving material from guidewire tube 236 may include cutting, ablating, machining, or otherwise removing material from guidewire tube 236 to create the smooth circumferential transition. Shaving material from guidewire tube 236 that sticks out of metal hypotube 228 at rapid exchange joint opening 230 may create a skived section 244 of guidewire tube 230 and reflowed polymer 238. Skived section 244 of guidewire tube 236 substantially removes any bump or protrusion by bringing a cross sectional profile of guidewire tube 236 and reflowed polymer 238 to substantially match an outer diameter of metal hypotube 228 and external coating or layer 242. Shaving material from guidewire tube 236 may also open an inner lumen of guidewire tube 236 that may have been covered by reflowed polymer 238 or other material when attaching guidewire tube 236 to metal hypotube 228.

The technique of FIG. 11 may additionally include assembling a neuromodulation element 112 (FIGS. 1-3) with metal hypotube 228 (410). When assembling neuromodulation element 112 (FIGS. 1-3) with metal hypotube 228, neuromodulation element 112 may include distal jacket 144 (FIGS. 2 and 3), one or more electrodes 146 (FIGS. 2 and 3), and one or more wires 348 (FIGS. 10A and 10B). A distal end or distal portion of guidewire tube 230 may extend to or beyond a distal end of metal hypotube 228 and be joined to a proximal end of distal shape-memory structure 142.

To assemble neuromodulation element 112 (FIGS. 1-3) with metal hypotube 228 (410), a mandrel or lasso may be extended through lumen 350 (FIGS. 10A and 10B) or metal hypotube 228 to protrude from a distal end of metal hypotube 228. The mandrel or lasso may include an element for coupling to proximal ends or proximal portions of one or more wires 348. The mandrel or lasso may be coupled to the proximal ends or proximal portions of one or more wires 348, and the mandrel or lasso pulled proximally to feed one or more wires 348 through lumen 350 of metal hypotube 228. A proximal end of neuromodulation element 112 also may be advanced toward a distal end of metal hypotube 228, e.g., as the one or more wires are fed through lumen 350. Distal jacket 144 may be advanced over distal shape-memory structure 142 and a distal portion of guidewire tube 230 to abut or overlap a distal portion of metal hypotube 228. Distal jacket 144 then may be joined to metal hypotube 228 (e.g., polymer outer jacket 242) using a reflowed polymer.

In other catheters where the shaft comprises more than a single metal hypotube, one or more wires 348 may need to be advanced through each of the multiple sections of the shaft. The present disclosure enables faster assembly of a neuromodulation catheter by using only a single metal hypotube 228 with varying flexibility along its length for the shaft of the catheter.

Catheters configured in accordance with at least some examples of the present technology may be well suited (e.g., with respect to sizing, flexibility, operational characteristics, and/or other attributes) for performing renal neuromodulation in human patients. Renal neuromodulation is the partial or complete incapacitation or other effective disruption of nerves of the kidneys (e.g., nerves terminating in the kidneys or in structures closely associated with the kidneys). In particular, renal neuromodulation may include inhibiting, reducing, and/or blocking neural communication along neural fibers (e.g., efferent and/or afferent neural fibers) of the kidneys. Such incapacitation may be long-term (e.g., permanent or for periods of months, years, or decades) or short-term (e.g., for periods of minutes, hours, days, or weeks). Renal neuromodulation is expected to contribute to the systemic reduction of sympathetic tone or drive and/or to benefit at least some specific organs and/or other bodily structures innervated by sympathetic nerves. Accordingly, renal neuromodulation is expected to be useful in treating clinical conditions associated with systemic sympathetic overactivity or hyperactivity, particularly conditions associated with central sympathetic overstimulation. For example, renal neuromodulation is expected to efficaciously treat hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, polycystic kidney disease, polycystic ovary syndrome, osteoporosis, erectile dysfunction, and sudden death, among other conditions.

Renal neuromodulation may be electrically-induced, thermally-induced, or induced in another suitable manner or combination of manners at one or more suitable treatment locations during a treatment procedure. The treatment location may be within or otherwise proximate to a renal lumen (e.g., a renal artery, a ureter, a renal pelvis, a major renal calyx, a minor renal calyx, or another suitable structure), and the treated tissue may include tissue at least proximate to a wall of the renal lumen. For example, with regard to a renal artery, a treatment procedure may include modulating nerves in the renal plexus, which lay intimately within or adjacent to the adventitia of the renal artery. Various suitable modifications may be made to the catheters described above to accommodate different treatment modalities. For example, the band electrodes may be replaced with transducers to facilitate transducer-based treatment modalities.

Renal neuromodulation may include an electrode-based treatment modality alone or in combination with another treatment modality. Electrode-based treatment may include delivering electricity and/or another form of energy to tissue at or near a treatment location to stimulate and/or heat the tissue in a manner that modulates neural function. For example, sufficiently stimulating and/or heating at least a portion of a sympathetic renal nerve may slow or potentially block conduction of neural signals to produce a prolonged or permanent reduction in renal sympathetic activity. A variety of suitable types of energy may be used to stimulate and/or heat tissue at or near a treatment location. For example, neuromodulation in accordance with examples of the present technology may include delivering RF energy and/or another suitable type of energy. An electrode used to deliver this energy may be used alone or with other electrodes in a multi-electrode array.

Heating effects of electrode-based treatment may include ablation and/or non-ablative alteration or damage (e.g., via sustained heating and/or resistive heating). For example, a treatment procedure may include raising the temperature of target neural fibers to a target temperature above a first threshold to achieve non-ablative alteration, or above a second, higher threshold to achieve ablation. The target temperature may be higher than about body temperature (e.g., about 37° C.) but less than about 45° C. for non-ablative alteration, and the target temperature may be higher than about 45° C. for ablation. Heating tissue to a temperature between about body temperature and about 45° C. may induce non-ablative alteration, for example, via moderate heating of target neural fibers or of luminal structures that perfuse the target neural fibers. In cases where luminal structures are affected, the target neural fibers may be denied perfusion resulting in necrosis of the neural tissue. Heating tissue to a target temperature higher than about 45° C. (e.g., higher than about 60° C.) may induce ablation, for example, via substantial heating of target neural fibers or of luminal structures that perfuse the target fibers. In some patients, it may be desirable to heat tissue to temperatures that are sufficient to ablate the target neural fibers or the luminal structures, but that are less than about 90° C. (e.g., less than about 85° C., less than about 80° C., or less than about 75° C.).

This disclosure is not intended to be exhaustive or to limit the present technology to the precise forms disclosed herein. Although specific examples are disclosed herein for illustrative purposes, various equivalent modifications are possible without deviating from the present technology, as those of ordinary skill in the relevant art will recognize. In some cases, well-known structures and functions have not been shown and/or described in detail to avoid unnecessarily obscuring the description of the examples of the present technology. Although steps of methods may be presented herein in a particular order, in alternative examples the steps may have another suitable order. Similarly, certain aspects of the present technology disclosed in the context of particular examples may be combined or eliminated in other examples. Furthermore, while advantages associated with certain examples may have been disclosed in the context of those examples, other examples may also exhibit such advantages, and not all examples need necessarily exhibit such advantages or other advantages disclosed herein to fall within the scope of the present technology. Accordingly, this disclosure and associated technology may encompass other examples not expressly shown and/or described herein.

Throughout this disclosure, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising” and the like are used throughout this disclosure to mean including at least the recited feature(s) such that any greater number of the same feature(s) and/or one or more additional types of features are not precluded. Directional terms, such as “upper,” “lower,” “front,” “back,” “vertical,” and “horizontal,” may be used herein to express and clarify the relationship between various elements. It should be understood that such terms do not denote absolute orientation. Reference herein to “one example,” “an example,” or similar formulations means that a particular feature, structure, operation, or characteristic described in connection with the example may be included in at least one example of the present technology. Thus, the appearances of such phrases or formulations herein are not necessarily all referring to the same example. Furthermore, various particular features, structures, operations, or characteristics may be combined in any suitable manner in one or more examples of the present technology.

Various aspects of the disclosure have been described. These and other aspects are within the scope of the following claims.

1. A catheter comprising:
a metal hypotube and a polymer outer jacket over the metal hypotube, wherein the metal hypotube is configured to bend with varying flexibility along a length of the metal hypotube, and wherein the metal hypotube defines a rapid exchange joint opening in an intermediate portion of the metal hypotube.

2. The catheter of claim 1, further comprising a guidewire tube comprising a proximal portion, wherein the proximal portion of the guidewire tube is positioned at least partially in the rapid exchange joint opening to allow a guidewire to pass from the inside of the metal hypotube distal to the rapid exchange joint opening to the outside of the catheter proximal of the rapid exchange joint opening.

3. The catheter of claim 2, wherein the guidewire tube comprises a skived section defining a smooth diameter transition between the polymer outer jacket and a location where a portion of the guidewire tube extends into the rapid exchange joint opening, and wherein the proximal portion of the guidewire tube is attached to the metal hypotube.

4. The catheter of claim 1, wherein the metal hypotube comprises:
a proximal portion configured to bend with a first flexibility along a length of the proximal portion;
the intermediate portion configured to bend with a second flexibility along a length of the intermediate portion; and
a distal portion configured to bend with a third flexibility along a length of the distal portion, wherein the first, second, and third flexibilities are different from one another.

5. The catheter of claim 4, wherein the second flexibility along the length of the intermediate portion comprises a gradually increasing flexibility along the length of the intermediate portion from a proximal end of the intermediate portion to a distal end of the intermediate portion.

6. The catheter of claim 4, wherein the intermediate portion is more flexible than the portion proximal, and the distal portion is more flexible than the intermediate portion.

7. The catheter of claim 1, wherein the metal hypotube comprises a tube wall, and wherein the tube wall comprises a plurality of holes in one or more patterns configured to allow the metal hypotube to bend with varying flexibility.

8. The catheter of claim 7, wherein the metal hypotube comprises:
a proximal portion, wherein the tube wall of the proximal portion of the metal hypotube comprises a plurality of holes in a first pattern configured to allow the proximal portion of the metal hypotube to bend with a first bending radius;
the intermediate portion, wherein the tube wall of the intermediate portion of the metal hypotube comprises a plurality of holes in a second pattern configured to allow the intermediate portion of the metal hypotube to bend with a second bending radius, wherein the second bending radius is smaller than the first bending radius; and
a distal portion, wherein the tube wall of the distal portion of the metal hypotube comprises a plurality of holes in a third pattern configured to allow the distal portion of the metal hypotube to bend with a third bending radius, wherein the third bending radius is smaller than the second bending radius.

9. The catheter of claim 1, wherein the catheter further comprises:
one or more electrodes;
one or more wires, each wire electrically coupled to an electrode of the one or more electrodes; and
an electrically insulative polymer disposed within an inner lumen of the metal hypotube proximal of the rapid exchange joint opening, wherein the electrically insulative polymer defines an inner lumen, wherein the one or more wires are positioned within the inner lumen of the electrically insulative polymer.

10. The catheter of claim 1, wherein the metal hypotube comprises a distal end, a proximal end, and a length therebetween, and the rapid exchange joint opening is positioned along the length of the metal hypotube between sixty percent and ninety percent of the length from the proximal end to the distal end.

11. A method for manufacturing a catheter comprising:
cutting a rapid exchange joint opening in an intermediate portion of a metal hypotube and a polymer jacket over the metal hypotube, wherein,
the rapid exchange joint opening facilitates formation of a rapid exchange joint; and
the metal hypotube is configured to bend with varying flexibility along a length of the metal hypotube.

12. The method of claim 11, further comprising positioning a proximal portion of a guidewire tube at least partially in the rapid exchange joint opening to allow a guidewire to pass from the inside of the metal hypotube distal to the rapid exchange joint opening to the outside of the catheter proximal of the rapid exchange joint opening.

13. The method of claim 12, further comprising:
attaching the guidewire tube to the metal hypotube; and
shaving material from the guidewire tube to create a smooth circumferential transition between a polymer outer jacket over the metal hypotube and a location where a portion of the guidewire tube extends outward from the metal hypotube.

14. The method of claim 11, further comprising:
forming a proximal portion of the metal hypotube, wherein the proximal portion is configured to bend with a first flexibility along a length of the proximal portion;
forming the intermediate portion of the metal hypotube, wherein the intermediate portion is configured to bend with a second flexibility along a length of the intermediate portion; and
forming a distal portion of the metal hypotube, wherein the distal portion is configured to bend with a third flexibility along a length of the distal portion, wherein the first, second, and third flexibilities are different from one another.

15. The method of claim 14, wherein the second flexibility comprises a gradually increasing flexibility along the length of the intermediate portion from a proximal end of the intermediate portion to a distal end of the intermediate portion.

16. The method of claim 11, further comprising laser-cutting a plurality of holes in one or more patterns in a tube wall of the metal hypotube, wherein the plurality of holes in one or more patterns are configured to allow the metal hypotube to bend with varying flexibility along a length of the metal hypotube.

17. The method of claim 16, wherein the metal hypotube comprises a proximal portion, an intermediate portion, and a distal portion, and wherein the method further comprises:
laser-cutting a plurality of holes on the tube wall of the proximal portion of the metal hypotube in a first pattern configured to allow the proximal portion of the metal hypotube to bend with a first bending radius;
laser-cutting a plurality of holes on the tube wall of the intermediate portion of the metal hypotube in a second pattern configured to allow the intermediate portion of the metal hypotube to bend with a second bending radius, wherein the second bending radius is smaller than the first bending radius; and
laser-cutting a plurality of holes on the tube wall of the distal portion of the metal hypotube in a third pattern configured to allow the distal portion of the metal hypotube to bend with a third bending radius, wherein the third bending radius is smaller than the second bending radius.

18. The method of claim 11, wherein cutting the rapid exchange joint opening in the metal hypotube comprises cutting the rapid exchange joint opening with a width no greater than eighty percent of an inner diameter of the metal hypotube.

19. The method of claim 11, further comprising positioning one or more wires of a distal assembly of the catheter through an inner lumen of an electrically insulative polymer, wherein the electrically insulative polymer is disposed within an inner lumen of the metal hypotube proximal of the rapid exchange joint opening.

20. The method of claim 11, wherein the metal hypotube comprises a distal end, a proximal end, and a length therebetween, and the rapid exchange joint opening is positioned along the length of the metal hypotube between sixty percent and ninety percent of the length from the proximal end to the distal end.
